(19) United States
(12) Patent Application Publication
(10) Pub. No.: US 2018/0177704 A1
(43) Pub. Date: Jun. 28, 2018

(54) COMPOSITIONS COMPRISING FRUIT ENZYME SUITABLE FOR ALTERING THE COLOR OF HAIR

(71) Applicant: L'OREAL, Paris (FR)

(72) Inventor: Michael DEGEORGE, Old Bridge, NJ (US)

(73) Assignee: L'OREAL, Paris (FR)

(21) Appl. No.: 15/392,924

(22) Filed: Dec. 28, 2016

(51) Int. Cl. A61K 8/66; A61Q 5/10; A61Q 5/08; A61K 8/22; A61K 8/97; A61K 8/23; A61K 8/41

(52) U.S. Cl. CPC A61K 8/66; A61Q 5/10; A61Q 5/08; A61K 8/22; A61K 2800/4324; A61K 8/23; A61K 8/41; A61K 2800/88; A61K 8/97

(57) ABSTRACT

Provided are hair color-altering compositions which comprise a fruit enzyme selected from the group consisting of bromelain, papain, and combinations thereof; and an oxidizing agent or hair colorant compound selected from the group consisting of oxidation dyes, direct dyes, pigments, and mixtures thereof. Also provided are kits and methods of using said compositions.

### TECHNICAL FIELD

The present disclosure generally relates to compositions for use in bleaching and/or coloring the hair.

### BACKGROUND

The process of changing the color of hair, can involve depositing an artificial color onto the hair which provides a different shade or color to the hair, and/or lifting the color of the hair, such as lightening the color of dark hair to lighter shades.

In general, hair lightening or color lifting compositions and hair dyeing compositions possess an alkalinity such that these compositions have a pH value of above 7, typically being at pH 9 and above, and may generally require the presence of an alkalizing agent such as ammonia or an ammonia gas-generating compound and/or an amine or ammonium-based compound in amounts sufficient to make such compositions alkaline. This alkalizing agent causes the hair shaft to swell, thus allowing active agents into the hair.

Imparting a color change or color effect on hair can be done using permanent, demi-permanent, and semi-permanent or temporary hair coloring products. Conventional permanent hair coloring products are dye compositions comprising oxidation dye precursors, which are also known as primary intermediates or couplers. These oxidation dye precursors are colorless or weakly colored compounds which, when combined with oxidizing products, give rise to colored complexes by a process of oxidative condensation. The oxidizing products (i.e., developers) conventionally use peroxides such as hydrogen peroxide as oxidizing agents. Such permanent hair color products also contain ammonia or other alkalizing agents such as monoethanolamine (MEA) which causes the hair shaft to swell, thus allowing the small oxidative dye molecules to penetrate the cuticle and cortex before the oxidation condensation process is completed. The resulting larger-sized colored complexes from the oxidative reaction are then trapped inside the hair fiber, thereby permanently altering the color of the hair. Demi-permanent dyeing also utilizes oxidation dye precursors, but are often used with a low volume developer (e.g., 2-3% hydrogen peroxide), as well as MEA.

Semi-permanent dyeing uses direct dyes, which are nonionic or ionic dyes and colored compounds capable of producing a more or less pronounced change of the natural color of the hair, resistant to shampoo-washing several times. These dyes may or may not be used in the presence of an oxidizing agent. In contrast with oxidation dye precursors, a direct dye is a relatively voluminous molecule that does not penetrate easily into the core of the fiber.

The process of lifting the color of hair, generally also requires the use of compositions that comprise at least one oxidizing agent (e.g., hydrogen peroxide). Such oxidizing agents enter the hair shaft and react with melanin in the hair, thereby lightening the color of the hair. Additional lift agents, such as persulfate salts, may be added to enhance the process.

However, some of these active ingredients can be harsh and cause adverse reactions in users. For example, persulfates can produce negative cutaneous and respiratory responses in some people. Thus, there is a desire to provide hair color/bleach compositions which utilize safer active ingredients while maintaining lift and color, or at least allow for reduced levels of conventional color/bleaching agents.

### SUMMARY

One aspect of the invention pertains to a hair color-altering composition comprising:
a. a fruit enzyme selected from the group consisting of bromelain, papain, and combinations thereof;
b. an oxidizing agent selected from the group consisting of persulfates, perborates, percarbonates, peracids, their salts, and combinations thereof;
c. an alkalizing agent.

In one or more embodiments, the composition contains less than 25% by weight oxidizing agent. In some embodiments, the oxidizing agent comprises persulfates and the composition contains less than 25% by weight of the persulfates. In one or more embodiments, the fruit enzyme is present as part of a fruit extract selected from the group consisting of papaya extract, pineapple extract, and combinations thereof. In some embodiments, the fruit extract is present in an amount of from about 1% to about 30% by weight. In one or more embodiments, the composition has a pH of greater than about 7. In some embodiments, the alkalizing agent is selected from the group consisting of NH₄OH, monoethanolamine, and combinations thereof, wherein the alkalizing agent is present in an amount of greater than about 0 to about 20% by weight of the composition.

A second aspect of the invention pertains to a hair color-altering composition comprising:
a. from about 1% to about 30% by weight of a fruit enzyme selected from the group consisting of bromelain, papain, and combinations thereof;
b. a hair colorant compound comprising an oxidation dye.

In one or more embodiments, the hair colorant compound comprises an oxidation dye selected from the group consisting of para-phenylenediamines, bis(phenyl)alkylenediamines, para-aminophenols, ortho-aminophenols, heterocyclic bases, the addition salts thereof, and combinations thereof. In some embodiments, the fruit enzyme is present as part of a fruit extract selected from the group consisting of papaya extract, pineapple extract, and combinations thereof. In one or more embodiments, the fruit extract is present in an amount of from about 1% to about 30% by weight. In some embodiments, the composition has a pH of greater than about 7. In one or more embodiments, the composition further comprises an alkalizing agent selected from the group consisting of NH₄OH, monoethanolamine, and combinations thereof, wherein the alkalizing agent is present in an amount of greater than about 0 to about 20% by weight of the composition.

A third aspect of the invention pertains to a hair color-altering composition comprising:
a. a fruit enzyme selected from the group consisting of bromelain, papain, and combinations thereof;
b. a hair colorant compound selected from the group consisting of direct dyes, pigments, and mixtures thereof.

In some embodiments, the hair colorant compound is a direct dye selected from the group consisting of azo direct dyes, (poly)methine dyes, carbonyl dyes, azine dyes, nitro(hetero)aryl dyes, tri(hetero)arylmethane dyes, porphyrin dyes, phthalocyanin dyes, cationic direct dyes, natural direct dyes and combinations thereof. In one or more embodiments, the fruit enzyme is present as part of a fruit extract selected from the group consisting of papaya extract, pineapple extract, and combinations thereof. In some embodiments, the fruit extract is present in an amount of from about 1% to about 30% by weight. In one or more embodiments, the composition has a pH of greater than about 7. In some embodiments, the composition further comprises an alkalizing agent selected from the group consisting of NH₄OH, monoethanolamine, and combinations thereof, wherein the alkalizing agent is present in an amount of greater than about 0 to about 20% by weight of the composition.

A fourth aspect of the invention pertains to a kit comprising
a. a first container comprising any of the hair color-altering composition described herein; and
b. a second container comprising a developer comprising hydrogen peroxide.

Yet another aspect of the invention pertains to a method of altering the color of human hair. In one or more embodiments, the method comprises applying to the hair the hair any of the color-altering compositions described above.

### BRIEF DESCRIPTION OF FIGURES

FIGS. 1A-B show the level of lift by a comparative bleach formula and several formulas according to one or more embodiments of the invention; and

FIGS. 2A-B show the effect of two comparative color formulas and several formulas according to one or more embodiments of the invention.

### DETAILED DESCRIPTION

Other than in the operating examples, or where otherwise indicated, all numbers expressing quantities of ingredients and/or reaction conditions are to be understood as being modified in all instances by the term “about” which can encompass ±10%, ±8%, ±6%, ±5%, ±4%, ±3%, ±2%, ±1%, or ±0.5%.

All numbers expressing pH values are to be understood as being modified in all instances by the term “about” which encompasses up to ±3%.

“At least one” as used herein means one or more and thus includes individual components as well as mixtures/combinations.

The term “altering the color” or “color-altering” as used herein may refer to lifting or lightening the color of hair. It can also refer to dyeing or coloring hair or depositing color onto the hair. In certain instances, it refers to lifting or lightening the color of hair and depositing color onto the hair in one treatment.

“Substituted,” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalkyl groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, and polysiloxane groups. The substituent(s) may be further substituted.

The term “neutralized” as used herein is intended to mean that the 3-butoxypropylamine is protonated with a H⁺ (proton) coming from the diacid(s).

The term “substantially free of (a component)” as defined herein means that the system or composition contains no appreciable amount of the component, for example, no more than about 1% by weight, no more than about 0.5% by weight, or no more than about 0.3% by weight, such as no more than about 0.1% by weight, based on the weight of the composition.

The term “free” or “completely free of (a component)” as defined herein means that the composition does not contain the component in any measurable degree by standard means.

### Color-Altering Compositions

As described herein, the disclosure relates to hair color-altering compositions comprising a fruit enzyme, particularly a fruit enzyme derived from papaya and/or pineapple. It has been surprisingly discovered that such compositions provide good color and/or lightening performance, while at the same time allowing for at least reduced levels of certain conventional actives (e.g., persulfates). The compositions described herein may also provide improved scalp comfort. While not wishing to be bound to any particular theory, it is thought that the fruit enzymes enhance lift and/or coloring by denaturing the protein.

In various exemplary embodiments, the color-altering composition may comprise an agent for bleaching the hair. For example, the color-altering composition may be formed by combining, in a cosmetically acceptable carrier, a bleach composition comprising at least one oxidizing agent chosen from persulfates, perborates, percarbonates, peracids, bromates, their salts and mixtures thereof, and a developer composition comprising hydrogen peroxide. In other embodiments, the oxidizing agent and developer may be separate. In yet further embodiments, the color-altering composition may comprise at least one colorant compound chosen from oxidative dye precursors, direct dyes, pigments, and mixtures thereof.

### Fruit Enzyme

According to one or more embodiments, the color-altering compositions may comprise a fruit enzyme. In some embodiments, the fruit enzyme may be in the form of an additive added to a ready-to-use hair color-altering composition. While not wishing to be bound to any particular theory, it is thought that such fruit enzymes help to denatures hair as it is being color-altered. This helps the color-alteration process, thereby allowing for reduced amounts of conventional actives.

In one or more embodiments, the fruit enzyme is present in the form of a fruit extract. In further embodiments, the fruit extract comprises from about 1, 2, 3, 4, 5, 10, 15, 20, 25, 30, 35, 40, 45, 50, 55, 60, 65, 70, 75, 80, 85, 90, or 95 to about 5, 10, 15, 20, 25, 30, 35, 40, 45, 50, 55, 60, 65, 70, 75, 80, 85, 90, 91, 92, 93, 94, 95, 96, 97, 98, 99% enzyme by weight.

In some embodiments, the fruit enzyme is selected from a fruit enzyme derived from a papaya (e.g., Carica papaya or Vasconcellea cundinamarcensis). In further embodiments, the fruit enzyme derived from a papaya is papain. Papain is a cysteine protease having broad proteolytic activity. The papain may be present in a fruit extract with other substances.

In some embodiments, the fruit enzyme is selected from a fruit enzyme derived from pineapple (e.g., Ananas comosus). In further embodiments the fruit enzyme is bromelain, a cysteine endopeptidase. Bromelain may be extracted from several part of the pineapple plant, and in some embodiments from the stem and/or fruit. The bromelain may be present in an extract with other substances.

In one or more embodiments, the fruit enzyme is present in an amount of greater than 0 to about 20% by weight of the composition. In some embodiments, the fruit enzyme is present in an amount of from about 0.5, 1, 1.5, 2, 2.5, 3 or 5% to about 5, 12, 15, 20, 25 or 30% by weight of the composition. In such embodiments, the amounts may refer to fruit extract or to the fruit enzyme itself. In one or more embodiments, the fruit enzyme portion of the composition comprises more than one fruit enzyme. For example, the composition may comprise papain and bromelain. In such cases, the amounts described above correspond to the total amount of fruit enzyme present in the composition.

### Bleaching Agent

According to various embodiments, the color-altering composition may comprise, in a cosmetically acceptable carrier, at least one oxidizing agent chosen from peroxides, persulfates, perborates, percarbonates, peracids, bromates, sulfates, their salts and mixtures thereof. The at least one oxidizing agent may, optionally, be water-soluble.

Optional peroxides useful herein include, for example, hydrogen peroxide, magnesium peroxide, PVP-peroxide, calcium peroxide, and sodium peroxide.

Exemplary, non-limiting persulfates include potassium persulfate, sodium persulfate, and ammonium persulfate. In various embodiments, exemplary oxidizing agents may be chosen from sodium perborate and sodium percarbonate. In further embodiments, exemplary peracids may be chosen from organic peracids having the general formula (I):

R—C(O)OOH (I)

wherein, in formula (I), R is chosen from saturated or unsaturated, substituted or unsubstituted, straight or branched chain, alkyl, aryl or alkaryl groups having from 1 to 22 carbon atoms. In at least some exemplary embodiments, mixtures of two or more oxidizing agents chosen from persulfates, perborates, percarbonates, peracids, bromates, and salts thereof, may be chosen.

In various embodiments, the at least one bleaching agent is chosen from alkali metal salts of perborates, percarbonates, bromates, and persulfates, such as, for example, ammonium, sodium, and potassium salts.

The at least one oxidizing agent of the bleach compositions according to various embodiments of the disclosure is utilized in an amount sufficient to lighten or “bleach” hair. By way of example only, the at least one oxidizing agent of the bleach composition may be present in an amount ranging from about 10% by weight to about 100% by weight, such as from about 20% to about 90% by weight, from about 30% to about 80% by weight, or from about 40% to about 75% by weight, based on the total weight of the bleach composition. In further embodiments, the at least one oxidizing agent of the bleach composition may be present in an amount ranging from about 5% to about 50%, such as about 10% to about 45%, or about 15% to about 40%. In one exemplary embodiment, the at least one oxidizing agent of the bleach composition may be present in an amount of at least 40% by weight, based on the total weight of the bleach composition.

One advantage of the compositions described herein, however, is that the presence of fruit enzyme can allow for less oxidizing agents than conventionally used. Thus, in some embodiments, the amount of oxidizing agent present in the hair color-altering composition ranges from greater than 0 or from 1, 2 or 5 to about 8, 10, 11, 12, 13, 14, 15, 16, 17, 18, 19, 20, 25 or 30% by weight.

### Bleach Composition

When the color-altering composition comprises separate bleach and developer compositions, the bleach composition may comprise at least one oxidizing agent chosen from persulfates, perborates, percarbonates, peracids, bromates, sulfates, their salts, and mixtures thereof, such as those described above. In various embodiments, the at least one oxidizing agent is chosen from alkali metal salts of perborates, percarbonates, bromates, sulfates, and persulfates, such as, for example, ammonium, sodium, and potassium salts. The bleach composition may also optionally comprise a cosmetically acceptable carrier.

The at least one oxidizing agent of the bleach compositions according to various embodiments of the disclosure is utilized in an amount sufficient to lighten or “bleach” hair. By way of example only, the at least one oxidizing agent of the bleach composition may be present in an amount ranging from about 10% by weight to about 100% by weight, such as from about 20% to about 90% by weight, from about 30% to about 80% by weight, or from about 40% to about 75% by weight, based on the total weight of the bleach composition. In further embodiments, the at least one oxidizing agent of the bleach composition may be present in an amount ranging from about 5% to about 50%, such as about 10% to about 45%, or about 15% to about 40%. In one exemplary embodiment, the at least one oxidizing agent of the bleach composition may be present in an amount of at least 40% by weight, based on the total weight of the bleach composition.

One advantage of the compositions described herein, however, is that the presence of fruit enzyme can allow for less oxidizing agents than conventionally used. Thus, in some embodiments, the amount of oxidizing agent present in the hair color-altering composition ranges from greater than 0 or from 1, 2 or 5 to about 8, 10, 11, 12, 13, 14, 15, 16, 17, 18, 19, 20, 25 or 30% by weight.

The bleach composition may be in any form, such as, for example, in the form of a powder, gel, liquid, foam, lotion, cream, mousse, and emulsion.

In various exemplary embodiments, the bleach composition may be anhydrous. Optionally, water may be added as an activator, by mixing it with the bleach composition.

The bleach composition of the present invention may also contain acid and alkali pH adjusters, which are well known in the art in the cosmetic treatment of keratin fibers, such as hair. Such pH adjusters include, but are not limited to, sodium metasilicate, silicate compounds, citric acid, ascorbic acid, and carbonate compounds.

The pH adjusters may, in various embodiments, be present in the bleach composition in an amount effective to provide the color-altering composition with a pH ranging from about 7 to about 11 when the bleach composition is combined with the developer composition. By way of example, the amount of pH adjuster may be present, in various embodiments, in an amount of at least about 0.01%, such as at least about 0.1%, at least about 0.2%, or at least about 0.5%.

According to one exemplary embodiment, the bleach composition is alkaline, with the pH ranging from about 7 to about 11. According to a further exemplary embodiment, the bleach composition has a pH higher than about 7, 8 or 9.

When the bleach composition is in powder form, the pH may be measured in a 1% solution in water.

Colorants may also optionally be present in the bleach compositions described herein. The colorants useful according to various embodiments of the disclosure are those colorants that are stable in the bleach composition, and can impart additional toning and coloring to hair. Exemplary hair colorants include, but are not limited to, pigments, liposoluble dyes, direct dyes, nacreous pigments, pearling agents, leuco dyes, optical lightening colorants, natural colorants and optically-variable pigments.

Lightening or lifting the color of the hair is typically evaluated by the variation in tone height before and after the application of a hair color-altering composition onto hair. This variation corresponds to the degree or level of lightening or lift. The notion of “tone” is based on the classification of the natural shades, one tone separating each shade from the shade immediately following or preceding it, which is well known to hairstyling professionals. The tone heights or levels range from 1 (black) to 10 (light blond), one unit corresponding to one tone; thus, the higher the number, the lighter the shade or the greater the degree of lift.

### Developer Composition

When the color-altering composition comprises a separate developer compositions, the developer composition may comprise hydrogen peroxide. The developer composition may also optionally comprise a cosmetically acceptable carrier.

In various exemplary embodiments, hydrogen peroxide is present in an amount of at least about 1% by weight, based on the total weight of the developer composition. In further embodiments, hydrogen peroxide is present in an amount ranging from about 0.1% to about 80% by weight, such as from about 1.0% to about 75% by weight, or from about 2% to about 10% by weight, based on the total weight of the developer composition. In further exemplary embodiments, the hydrogen peroxide may be present in the developer composition in an amount ranging from about 2% to about 25%, such as about 4% to about 20%, about 6% to about 15%, or about 7% to about 10%.

The cosmetically acceptable carrier of the developer composition may, for example, be present in an amount ranging from about 0.5% to about 99% by weight, such as from about 5% to about 95% by weight, relative to the total weight of the developer composition.

The pH of the developer composition can range from about 1 to about 5, such as from about 2 to about 4, and it may be adjusted to the desired value using pH adjusters that are well known in the art in the cosmetic treatment of keratin fibers, including, for example, those described herein.

The developer composition may be in the form of a powder, gel, liquid, foam, lotion, cream, mousse, and emulsion.

According to various exemplary embodiments, the developer composition may be anhydrous. Optionally, water may be added as an activator, by mixing it with the developer composition.

The developer composition may, in various embodiments, comprise additional components such as, for example, at least one auxiliary ingredient chosen from rheology-modifying agents, chelants, fatty substances, ceramides, alkoxyaminosilicones, and silanes, and any other component known in the art to be useful in a developer composition.

In at least one exemplary embodiment, the bleach composition may be mixed with the developer composition to form the color-altering composition right before (e.g. within a few minutes before) applying the color-altering composition onto the hair.

In one exemplary embodiment, the bleach composition and developer composition may be combined to form the lightening composition in a ratio of bleach composition to developer composition ranging from about 1:1 to about 1:5, such as from about 1:1 to about 1:2, or about 1:2 to about 1:4.

### Coloring Compounds

As described herein, in various exemplary and non-limiting embodiments, color-altering compositions may optionally comprise at least one colorant compound chosen from oxidation dyes, direct dyes, pigments, and mixtures thereof.

The oxidation dyes are generally chosen from one or more oxidation bases optionally combined with one or more couplers.

By way of example, the oxidation bases may be chosen from para-phenylenediamines, bis(phenyl)alkylenediamines, para-aminophenols, ortho-aminophenols and heterocyclic bases, and the addition salts thereof.

Among the para-phenylenediamines that may be mentioned, for example, are para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(3-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the addition salts thereof with an acid.

Among the para-phenylenediamines mentioned above, para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine, and the addition salts thereof with an acid, are particularly preferred.

Among the bis(phenyl)alkylenediamines that may be mentioned, for example, are N,N′-bis(β-hydroxyethyl)-N,N′-bis(4′-aminophenyl)-1,3-diaminopropanol, N,N′-bis(3-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the addition salts thereof.

Among the para-aminophenols that may be mentioned, for example, are para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, and the addition salts thereof with an acid.

Among the ortho-aminophenols that may be mentioned, for example, are 2-aminophenol, 2-amino-5-methylphenol, 2-amino-6-methylphenol and 5-acetamido-2-aminophenol, and the addition salts thereof.

Among the heterocyclic bases that may be mentioned, for example, are pyridine derivatives, pyrimidine derivatives and pyrazole derivatives.

Among the pyridine derivatives that may be mentioned are the compounds described, for example, in patents GB 1 026 978 and GB 1 153 196, for instance 2,5-diaminopyridine, 2-(4-methoxyphenyl)amino-3-aminopyridine and 3,4-diaminopyridine, and the addition salts thereof.

Other pyridine oxidation bases can include the 3-aminopyrazolo[1,5-a]pyridine oxidation bases or the addition salts thereof described, for example, in patent application FR 2 801 308. Examples that may be mentioned include pyrazolo[1,5-a]pyrid-3-ylamine, 2-acetylaminopyrazolo[1,5-a]pyrid-3-ylamine, 2-morpholin-4-ylpyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-morpholin-4-ylpyrazolo[1,5-a]pyrid-3-ylamine, pyrazolo[1,5-a]pyridine-3,5-diamine, 5-morpholin-4-ylpyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-□-hydroxyethoxy-3-amino-pyrazolo[1,5-a]pyridine; 2-(4-diméthylpyperazinium-1-yl)-3-amino-pyrazolo[1,5-a]pyridine; and the addition salts thereof.

More particularly, oxidation bases can be selected from 3-aminopyrazolo-[1,5-a]-pyridines and preferably substituted on carbon atom 2 by:

one (di)(C1-C6)(alkyl)amino group wherein said alkyl group can be substituted by at least one hydroxy, amino, imidazolium group;

one heterocycloalkyl group containing from 5 to 7 members chain, and from 1 to 3 heteroatomes, potentially cationic, potentially substituted by one or more (C1-C6)alkyl, such as di(C1-C4)alkylpiperazinium; or

one (C1-C6)alkoxy potentially substituted by one or more hydroxy groups such as α-hydroxyalkoxy, and the addition salts thereof.

Among the pyrimidine derivatives that may be mentioned are the compounds described, for example, in the patents DE 2359399; JP 88-169571; JP 05-63124; EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and their addition salts and their tautomeric forms, when a tautomeric equilibrium exists.

Among the pyrazole derivatives that may be mentioned are the compounds described in the patents DE 3843892, DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)-pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole, and the addition salts thereof. 4,5-Diamino-1-(β-methoxyethyl)pyrazole may also be used.

A 4,5-diaminopyrazole will preferably be used, and even more preferentially 4,5-diamino-1-(β-hydroxyethyl)pyrazole and/or a salt thereof.

Pyrazole derivatives that may also be mentioned include diamino-N,N-dihydro-pyrazolopyrazolones and especially those described in patent application FR-A-2 886 136, such as the following compounds and the addition salts thereof: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-di-(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one, 2,3-diamino-6-hydroxy-6,7-dihydro-1H,5H-pyrazolo[1,2-a]pyrazol-1-one.

2,3-Diamino-6,7-dihydro-1H,5H-pyrazolo[1,2-a]pyrazol-1-one and/or a salt thereof will preferably be used.

4,5-Diamino-1-(β-hydroxyethyl)pyrazole and/or 2,3-diamino-6,7-dihydro-1H,5H-pyrazolo[1,2-a]pyrazol-1-one and/or a salt thereof will preferentially be used as heterocyclic bases.

Compositions may optionally further comprise one or more couplers advantageously chosen from those conventionally used in the dyeing or coloring of keratinous substrates.

Among these couplers, mention may be made especially of meta-phenylenediamines, meta-aminophenols, meta-diphenols, naphthalene-based couplers and heterocyclic couplers, and also the addition salts thereof.

Mention may be made, for example, of 2-methyl-5-aminophenol, 5-N-(ß-hydroxyethyl)amino-2-methylphenol, 3-aminophenol, 5-amino-6-chloro-o-cresol (3-amino-2-chloro-6-methylphenol), 1,3-dihydroxybenzene, 1,3-dihydroxy-2-methylbenzene, 4-chloro-1,3-dihydroxybenzene, 2,4-diamino-1-(ß-hydroxyethyloxy)benzene, 2-amino-4-(ß-hydroxy-ethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-ß-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzo-morpholine, 3,5-diamino-2,6-dimethoxypyridine, 1-N-(ß-hydroxyethyl)amino-3,4-methylenedioxybenzene, 2,6-bis(ß-hydroxyethylamino)toluene, 6-hydroxyindoline, 2,6-dihydroxy-4-methylpyridine, 1-H-3-methylpyrazol-5-one, 1-phenyl-3-methylpyrazol-5-one, 2,6-dimethylpyrazolo[1,5-b]-1,2,4-triazole, 2,6-dimethyl[3,2-c]-1,2,4-triazole and 6-methyl-pyrazolo[1,5-a]benzimidazole, the addition salts thereof with an acid, and mixtures thereof.

In general, the addition salts of the oxidation bases and couplers that may be used are chosen from the addition salts with an acid such as the hydrochlorides, hydrobromides, sulfates, citrates, succinates, tartrates, lactates, tosylates, benzenesulfonates, phosphates and acetates.

The oxidation base(s) may be present in an amount ranging from about 0.001% to 10% by weight, such as from about 0.005% to 5% by weight, relative to the total weight of the composition comprising the system in which it is present.

The coupler(s), if they are present, may be present in an amount ranging from about 0.001% to 10% by weight, such as from about 0.005% to 5% by weight, relative to the total weight of the system or composition comprising the system in which it is present.

Compositions according to embodiments of the disclosure may optionally comprise one or more synthetic or natural direct dyes, for example chosen from anionic and nonionic species, preferably cationic or nonionic species, either as sole dyes or in addition to the oxidation dye(s).

Examples of suitable direct dyes that may be mentioned include azo direct dyes; (poly)methine dyes such as cyanins, hemicyanins and styryls; carbonyl dyes; azine dyes; nitro(hetero)aryl dyes; tri(hetero)arylmethane dyes; porphyrin dyes; phthalocyanin dyes, and natural direct dyes, alone or as mixtures.

Preferably direct dyes are cationic direct dyes. Mention may be made of the hydrazono cationic dyes of formulas (Va) and (V′a), the azo cationic dyes (VIa) and (VI′a) and the diazo cationic dyes (VIIa) below:

Het⁺-C(R^{a})═N—N(R^{b})—Ar,An⁻ (Va)

Het⁺-N(R^{a})—N═C(R^{b})—Ar,An⁻ (V′a)

Het⁺-N═N—Ar,An⁻ (VIa)

Ar⁺-N═N—Ar″,An⁻ (VI′a) and

Het⁺-N═N—Ar′-N═N—Ar,An⁻ (VIIa)

in which formulas (Va), (V′a), (VIa), (VI′a) and (VIIa):
Het⁺ represents a cationic heteroaryl moiety, preferably bearing an endocyclic cationic charge, such as imidazolium, indolium or pyridinium, optionally substituted preferentially with one or more (C₁-C₈) alkyl groups such as methyl;
Ar⁺ representing an aryl moiety, such as phenyl or naphthyl, bearing an exocyclic cationic charge, preferentially ammonium, particularly tri(C₁-C₈)alkylammonium such as trimethylammonium;
Ar represents an aryl group, especially phenyl, which is optionally substituted, preferentially with one or more electron-donating groups such as i) optionally substituted (C₁-C₈)alkyl, ii) optionally substituted (C₁-C₈)alkoxy, iii) (di)(C₁-C₈)(alkyl)amino optionally substituted on the alkyl group(s) with a hydroxyl group, iv) aryl(C₁-C₈)alkylamino, v) optionally substituted N—(C₁-C₈)alkyl-N-aryl(C₁-C₈)alkylamino or alternatively Ar represents a julolidine group;
Ar′ is an optionally substituted divalent (hetero)arylene group such as phenylene, particularly para-phenylene, or naphthalene, which are optionally substituted, preferentially with one or more groups (C₁-C₈)alkyl, hydroxyl or (C₁-C₈)alkoxy;
Ar″ is an optionally substituted (hetero)aryl group such as phenyl or pyrazolyl, which are optionally substituted, preferentially with one or more groups (C₁-C₈)alkyl, hydroxyl, (di)(C₁-C₈)(alkyl)amino, (C₁-C₈)alkoxy or phenyl;
R^{a} and R^{b}, which may be identical or different, represent a hydrogen atom or a group (C₁-C₈)alkyl, which is optionally substituted, preferentially with a hydroxyl group; or alternatively the substituent R^{a} with a substituent of Het⁺ and/or R^{b} with a substituent of Ar and/or R^{a} with R^{b} form, together with the atoms that bear them, a (hetero)cycloalkyl;
particularly, R^{a} and R^{b} represent a hydrogen atom or a group (C₁-C₄)alkyl, which is optionally substituted with a hydroxyl group;
An⁻ represents an anionic counter-ion such as mesylate or halide.

In particular, mention may be made of the azo and hydrazono cationic dyes bearing an endocyclic cationic charge of formulae (Va), (V′a) and (VIa) as defined previously. More particularly mention may be made of those of formulae (Va), (V′a) and (VIa) derived from the dyes described in patent applications WO 95/15144, WO 95/01772 and EP-714954.

In various embodiments, the cationic part is derived from the following derivatives:

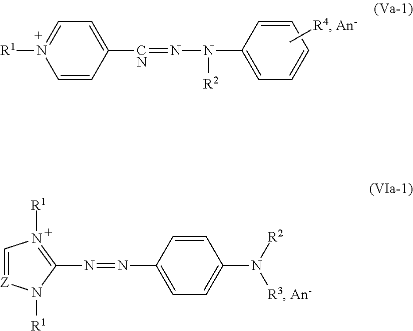

wherein in formulae (Va-1) and (VIa-1):
R¹ representing a (C₁-C₄) alkyl group such as methyl;
R² and R³, which are identical or different, represent a hydrogen atom or a (C₁-C₄)alkyl group, such as methyl; and
R⁴ represents a hydrogen atom or an electron-donating group such as optionally substituted (C₁-C₈)alkyl, optionally substituted (C₁-C₈)alkoxy, or (di)(C₁-C₈)(alkyl)amino optionally substituted on the alkyl group(s) with a hydroxyl group; particularly, R⁴ is a hydrogen atom,
Z represents a CH group or a nitrogen atom, preferentially CH;
An⁻ represents an anionic counter-ion such as mesylate or halide.

The dye of formulae (Va-1) and (VIa-1) can be chosen from Basic Red 51, Basic Yellow 87 and Basic Orange 31 or derivatives thereof:

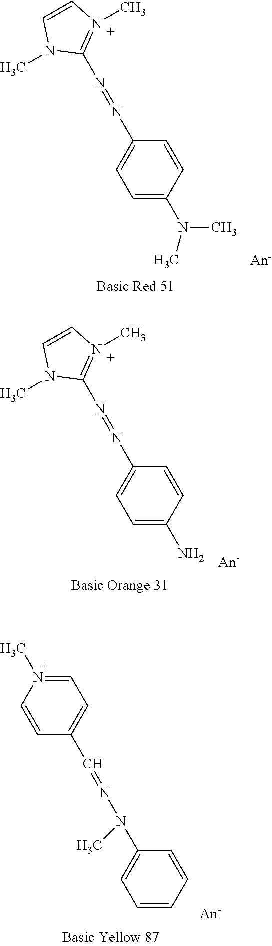

Among the natural direct dyes, mention may be made of lawsone, juglone, alizarin, purpurin, carminic acid, kermesic acid, purpurogallin, protocatechaldehyde, indigo, isatin, curcumin, spinulosin, apigenidin and orceins. Extracts or decoctions containing these natural dyes and in particular henna-based poultices or extracts may also be used. When they are present, the one or more direct dyes more particularly represent from about 0.001% to 10% by weight, such as from about 0.005% to 5% by weight, of the total weight of the system or composition comprising the system in which it is present.

The color-altering composition may also comprise a cosmetically acceptable carrier. The cosmetically acceptable carrier may, for example, be present in the color-altering composition in an amount ranging from about 1% to about 40% by weight, such as from about 5% to about 35% by weight, or about 10% to about 30% by weight of the color-altering composition.

### Alkalizing Agents

The hair color-altering composition may have a pH that is alkaline. Exemplary pH's include 7, 8, 9, 10, 11, 12, 13 or 14. In some embodiments, the pH of the hair color-altering composition may range from about 7, 8, or 9 to about 9, 10, 11 or 12.

The alkalinity of the hair color-altering composition may be derived from one or more alkalizing agents. In some embodiments, the alkalizing agent may ammonia or an ammonia gas-generating compound and/or an amine or ammonium-based compound in amounts sufficient to make such compositions alkaline. In further embodiments, the alkalizing agent may be selected from alkanolamines, such as monoethanolamine (MEA) and isopropanolamine. Alkalinity may be derived from ammonium compounds as well (e.g., NH4OH).

The one or more alkalizing agents may be present in amounts ranging from greater than about 0, or from 1, 2, 3, 4, 5, 10 to about 5, 10, 13, 15, 18, 20, 25 or 30% by weight of the total composition.

### Auxiliary Agents

Auxiliary ingredients may be added to the color-altering composition. Exemplary auxiliary ingredients useful in the color-altering composition according to various embodiments of the disclosure include, but are not limited to, rheology-modifying agents, bleach activators and co-bleach activators, direct dyes, chelants, fatty substances, ceramides, alkoxyaminosilicones, silanes, and lift-enhancing agents, such as nitrogen-containing compounds and metal catalyst compounds.

The color-altering composition may also contain acid and alkali pH adjusters, which are well known in the art in the cosmetic treatment of keratin fibers, such as hair. Such pH adjusters include, but are not limited to, sodium metasilicate, silicate compounds, citric acid, ascorbic acid, and carbonate compounds.

### Kits and Methods

Another aspect of the invention pertains to kits comprising the color-altering compositions described herein. For example, developer may be present in a separate container from the color-altering composition which comprises the fruit enzyme, oxidizing agent, hair colorant and/or oxidizing agent. In such embodiments, the developer and hair color-altering composition are combined just prior to use. The color-altering composition may, in some embodiments, be in a ready-to-use form.

Another aspect of the invention pertains to methods of using the color-altering compositions. The methods comprise applying the compositions described herein to human hair. The color-altering composition may be left on the hair for a period of time sufficient to achieve the desired alteration in hair tone. For example, the color-altering composition may be left on the hair for up to one hour, such as from about 3 minutes to about 45 minutes, from about 5 minutes to about 30 minutes, or from about 10 minutes to about 20 minutes. In further embodiments, the color-altering composition may be left on the hair for a period up to about 30 minutes, such as, for example, from about 1 to about 30 minutes, about 1 to about 10 minutes, or about 1 to about 5 minutes. One skilled in the art will, by considering various factors such as the starting and desired tones of the hair, be able to determine an appropriate amount of time to leave the color-altering composition on the hair in order to achieve the desired alternation in hair tone. By way of non-limiting example, various embodiments according to the disclosure may provide for an increase of 1 to 4 in the tone height of the hair.

In some embodiments, the color-altering composition may, optionally, be shampooed and/or rinsed off the hair.

### Exemplary Embodiments

In one embodiment, the invention pertains to a hair color-altering composition which is suitable as a bleaching composition. Such embodiment comprises
a. a fruit enzyme selected from the group consisting of bromelain, papain, and combinations thereof; and
b. an oxidizing agent selected from the group consisting of persulfates, perborates, percarbonates, peracids, their salts, and combinations thereof.

The color-altering composition may further comprise a developer (e.g., hydrogen peroxide) and/or alkalizing agent. In some embodiments, the developer may be in a separate container from the fruit enzyme, oxidizing agent and/or alkalizing agent. Any of the above embodiments may be combined with this exemplary embodiment.

In one embodiment, the invention pertains to a hair color-altering composition which is suitable as a hair color composition. Such embodiment comprises
a. a fruit enzyme selected from the group consisting of bromelain, papain, and combinations thereof; and
b. a hair colorant compound selected from the group consisting of direct dyes, pigments, oxidation dyes, and mixtures thereof.

The color-altering composition may further comprise a developer (e.g., hydrogen peroxide) and/or alkalizing agent. In some embodiments, the developer may be in a separate container from the fruit enzyme, oxidizing agent and/or alkalizing agent. Any of the above embodiments may be combined with this exemplary embodiment.

### EXAMPLES

The ingredient amounts in the composition/formulations described below are expressed in % by weight, based on the total weight of the composition, unless otherwise indicated.

### Example 1—Bleach Powder

One comparative and several inventive bleach powder formulas were prepared. The inventive formulas were based on the comparative but contained 5% (Inventive 2), 10% (Inventive 3), or 20% (Inventive 4) papaya extract or 5% (Inventive 5), 10% (Inventive 6), 20% (Inventive 7) pineapple extract. The inventive examples also had reduced amounts of potassium persulfate, ammonium sulfate and ammonium sulfate, and did not include fragrance or color. A detailed listing of the ingredients for Comparative 1 and Inventive 3 is shown in the ingredient list shown in Table 2 below. Inventives 2 and 4-7 are similar to Inventive 3 except with varying amounts of papaya or pineapple extract, with corn starch making up the remainder.

TABLE 2
Comparative Formula and Inventive Formulas Containing
Papaya Extract

| Ingredient | Comparative 1 | Inventive 3 | |
|---|---|---|---|
| Potassium | 36.00 | 18.00 | Oxidizer |
| Persulfate | | | |
| Sodium Silicate | 24.0 | 24.00 | Neutralizing |
| | | | Agent |
| Sodium | 11.5 | 5.75 | Oxidizer |
| Persulfate | | | |
| Ammonium | 5.00 | 2.50 | Oxidizer |
| Sulfate | | | |
| Sodium | 2.00 | 2.00 | Silica |
| Metasilicate | | | |
| Corn Starch | 6.25 | 25.70 | Filler |
| Sodium Lauryl | 4.00 | 4.00 | Detergent |
| Sulfate | | | |
| Hydrated Silica | 1.75 | 1.75 | Oil |
| | | | Absorbent |
| Thickeners | 3.50 | 3.50 | Thickening |
| EDTA | 0.80 | 0.80 | Chleating |
| Hydrogenated | 2.00 | 2.00 | Hydrating |
| Polydecene | | | Agent |
| Carica Papaya | 0.00 | 10.00 | Lift Active |
| (Papaya) Fruit | | | |
| Extract | | | |
| Color | 0.25 | 0.00 | Color |
| Fragrance | 1.00 | 0.00 | Fragrance |

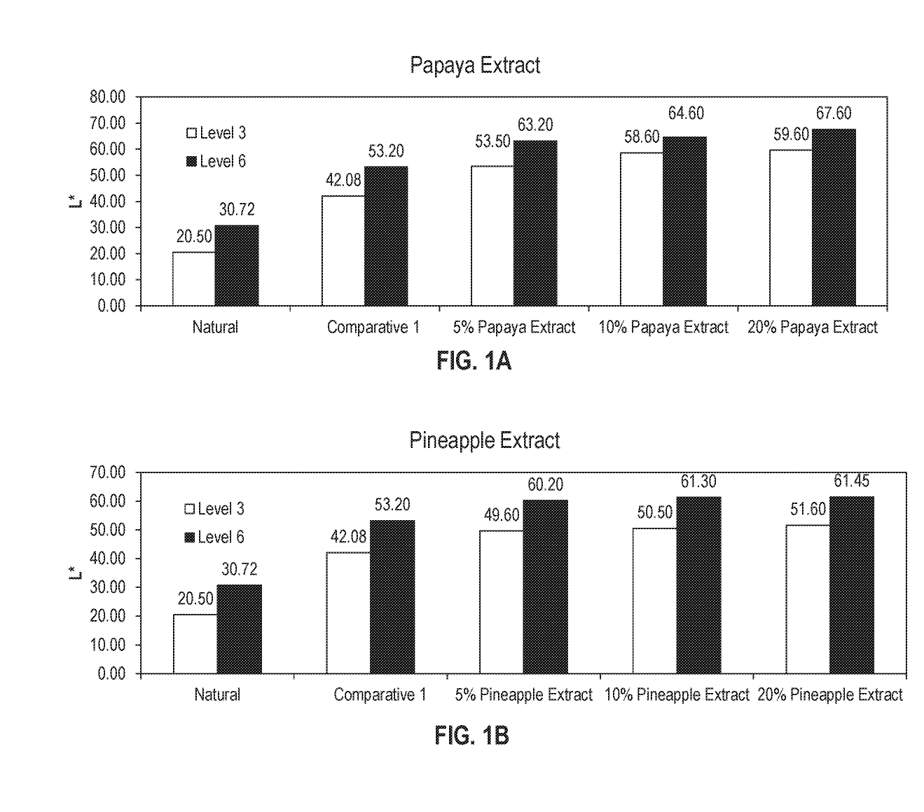

The bleach powder formulas were processed on hair swatches at room temperature using 12% hydrogen peroxide developer for 50 minutes. Each of the formulas was tested on hair swatches starting at either level 3 or level 6 hair lightness. The resulting level of lift is shown in FIGS. 1A-1B. As can be seen from the figures, the presence of papaya and pineapple extract exhibit actually increase the lift compared to the comparative. This is surprising because the comparative has much more active lightening agents than the inventive examples.

### Example 2—Hair Color: Demi-Permanent

Two demi-permanent color formulas (Comparative 8 and Inventive 9) were prepared. The detailed ingredient listing for Comparative 8 is shown in Table 3 below. Inventive 9 was the same as Comparative 8 except papaya extract was added to achieve 5% by weight (i.e., the papaya extract was an additive).

TABLE 3
Comparative Formula and Inventive Formula Containing
Papaya Extract

| INCI US Name | Comparative 8 |
|---|---|
| COCODIMONIUM HYDROXYPROPYL | 0.145 |
| HYDROLYZED RICE PROTEIN | |
| FRAGRANCE | 0.3 |
| CETYL HYDROXYETHYLCELLULOSE | 0.4 |
| MICA | 0.2115 |
| TITANIUM DIOXIDE | 0.0885 |
| ETHANOLAMINE | 0.2 |
| POTASSIUM SORBATE | 0.003 |
| POLYQUATERNIUM-6 | 0.8 |
| SODIUM BENZOATE | 0.003 |
| GLYCERIN | 3 |
| EDTA | 0.2 |
| CETEARYL ALCOHOL | 0.03 |
| WATER | 71.753 |
| SODIUM SULFATE | 0.03 |
| SODIUM SULFITE | 0.1 |
| PLANT SEED OIL | 0.2 |
| SURFACTANTS | 22.136 |
| ERYTHORBIC ACID | 0.4 |
| Total: | 100 |

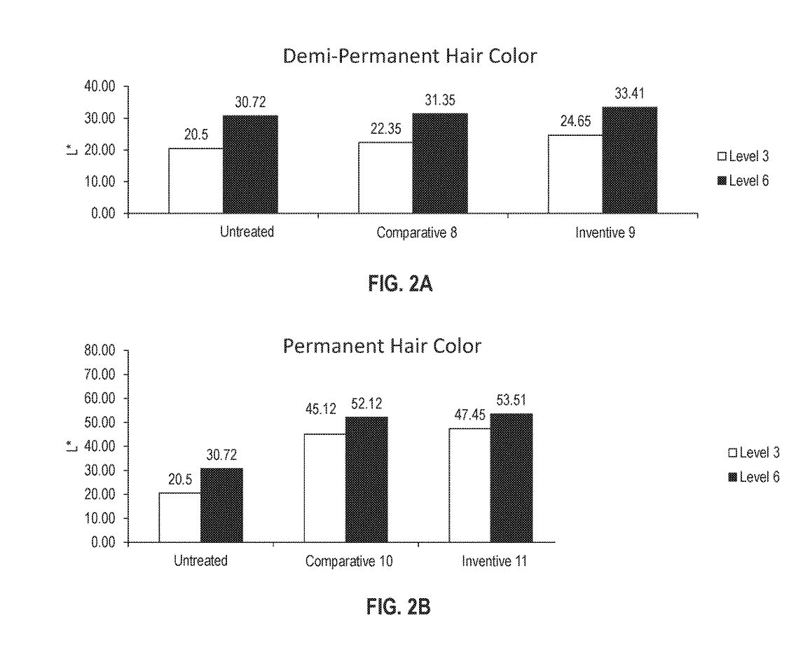

The demi-permanent color formulas were processed on hair swatches using 3% hydrogen peroxide developer for 20 minutes. Each of the formulas was tested on hair swatches starting at either level 3 or level 6 hair lightness. The resulting level of lift is shown in FIG. 2A. As can be seen from the figure, the presence of papaya extract increased the lift compared to the comparative example.

### Example 3—Hair Color: Permanent

Two permanent color formulas (Comparative 10 and Inventive 11) were prepared. The detailed ingredient listing for Comparative 10 is shown in Table 4 below. Inventive 11 was the same as Comparative 8 except papaya extract was added to achieve 5% by weight (i.e., the papaya extract was an additive).

TABLE 4
Comparative Formula and Inventive Formula Containing
Papaya Extract

| Ingredient | Comparative 10 |
|---|---|
| ETHANOLAMINE | 5.048 |
| ACRYLATES COPOLYMER | 1.05 |
| ASCORBIC ACID | 0.12 |
| GLYCERIN | 5 |
| EDTA | 0.2 |
| SODIUM METABISULFITE | 0.22 |
| WATER | 28.362 |
| MINERAL OIL | 60 |
| Total | 100 |

The permanent color formulas were processed on hair swatches using 30 volume hydrogen peroxide developer for 20 minutes. Each of the formulas was tested on hair swatches starting at either level 3 or level 6 hair lightness. The resulting level of lift is shown in FIG. 2B. As can be seen from the figure, the presence of papaya extract increased the lift compared to the comparative example.

### Example 4—Cysteic Acid Measurements in Bleach Formulations

One comparative (Comparative 12) and two inventive (Inventives 13 and 14) bleach formulas were prepared. Inventive 12 is similar to Comparative 11, but contains 10% by weight of papaya extract. Inventive 13 is similar to Inventive 12 in that it also contains papaya extract, but different from Inventive 12 and Comparative 11 in that Inventive 13 contains about half the amount of persulfate/sulfate. The detailed ingredient listing can be found in Table 5 below.

TABLE 5
Comparative Formula and Inventive Formula Containing
Papaya Extract

| RM | Comparative 11 | Inventive 12 | Inventive 13 |
|---|---|---|---|
| Colorant | 0.08 | 0.08 | 0.08 |
| Magnesium Carbonate | 9.43 | 9.43 | 9.43 |
| Hydroxide | | | |
| Sodium Silicate | 17.36 | 17.36 | 17.36 |
| Disodium EDTA | 0.98 | 0.98 | 0.98 |
| Panthenol | 0.11 | 0.11 | 0.11 |
| SODIUM | 2.48 | 2.48 | 2.48 |
| METASILICATE | | | |
| SODIUM LAURYL | 0.99 | 0.91 | 0.91 |
| SULFATE | | | |
| POTASSIUM | 49.61 | 49.61 | 24.80 |
| PERSULFATE | | | |
| AMMONIUM | 4.96 | 4.96 | 2.48 |
| PERSULFATE | | | |
| Deionized Water | 0.010 | 0.010 | 0.010 |
| Mineral Oil | 1.98 | 1.98 | 1.98 |
| Sodium Stearate | 9.92 | — | — |
| Guar Gum | 2.08 | 2.00 | 2.08 |
| Corn Starch | — | — | 27.28 |
| CARICA PAPAYA | — | 10.00 | 10.00 |
| (PAPAYA) FRUIT | | | |
| EXTRACT | | | |

The above formulas were applied to hair swatches and processed for 50 minutes with 20 volume developer. The cysteic acid after hair swatches were treated with the above formulas was measured. Cystine bridges, or disulfide bonds, are the strongest bonds present in the internal network of hair and play a key role in hair strength. Cosmetic treatments, such as bleaches, can permanently alter these bonds, causing the formation of cysteic acid. Since the formation of cysteic acid is an irreversible process, a lower measured concentration actually means a particular invention provided a protection benefit to the hair fiber and prevented and/or minimized this disulfide bond modification. Therefore, by measuring the concentration of cysteic acid in hair it is possible to assess fiber integrity.

Replicate samples of hair were hydrolyzed under acidic conditions for 16 hours at 110° C. Once hydrolyzed samples were pH adjusted to approximately 1.7 with a solution of lithium hydroxide and analyzed on a Hitachi amino acid analyzer, Model 8900. Amino acid standards obtained from Sigma Aldrich (Ref AAS 18) were utilized to calibrate the instrument and to calculate the concentration of amino acids for each of the treatment conditions.

Cysteic acid was measured on untreated hair, as well as after 1 and 3 applications of the above formulas. The results are shown in Table 6 below.

TABLE 6
Cysteic Acid Results

| | Control | Comparative | | |
|---|---|---|---|---|
| | (no treatment) | 12 | Inventive 13 | Inventive 14 |
| Cysteic | 0.5 | 4.8 | 3.1 | 3.4 |
| Acid (%) | | | | |
| After 1 | | | | |
| Application | | | | |
| Cysteic | 0.5 | 11.2 | 7.5 | 7.9 |
| Acid (%) | | | | |
| After 3 | | | | |
| Applications | | | | |

As can be seen from Table 6, the cysteic acid was greatly reduced in Inventive 13 as compared to Comparative 12 simply by the addition of papaya extract. What is even more surprising is that for Inventive 14, cysteic acid levels were reduced almost to the same level as Inventive 13, even though it had half of the amount of active agent compared to Comparative 12.

1. A hair color-altering composition comprising:
a. from about 0.5% to about 30% by weight of a fruit enzyme selected from the group consisting of bromelain, papain, and combinations thereof;
b. an oxidizing agent selected from the group consisting of persulfates, perborates, percarbonates, peracids, their salts, and combinations thereof;
c. an alkalizing agent; and
d. optionally, at least one colorant compound,
provided that if the hair color-altering composition includes colorant compound(s), the colorant compound(s) are selected from the group consisting of oxidative dyes, direct dyes, pigments, and mixtures thereof.

2. The hair color-altering composition of claim 1, wherein the composition contains less than 25% by weight oxidizing agent.

3. The hair color-altering composition of claim 1, wherein the oxidizing agent comprises persulfates and the composition contains less than 25% by weight of the persulfates.

4. The hair color-altering composition of claim 1, wherein the fruit enzyme is present as part of a fruit extract selected from the group consisting of papaya extract, pineapple extract, and combinations thereof.

5. (canceled)

6. The hair color-altering composition of claim 1, wherein the composition has a pH of greater than about 7.

7. The hair color-altering composition of claim 1, wherein the alkalizing agent is selected from the group consisting of NH₄OH, monoethanolamine, and combinations thereof, wherein the alkalizing agent is present in an amount of greater than about 0 to about 20% by weight of the composition.

8. A kit comprising:
a. a first container comprising the hair color-altering composition of claim 1; and
b. a second container comprising a developer comprising hydrogen peroxide.

9. A hair color-altering composition comprising:
a. from about 1% to about 30% by weight of a fruit enzyme selected from the group consisting of bromelain, papain, and combinations thereof;
b. an oxidation dye; and
c. optionally, at least one additional hair colorant compound,
provided that if the hair color-altering composition includes additional hair colorant compound(s), the additional hair colorant compound(s) are selected from the group consisting of oxidative dyes, direct dyes, pigments, and mixtures thereof.

10. The hair color-altering composition of claim 9, wherein the oxidation dye is selected from the group consisting of para-phenylenediamines, bis(phenyl)alkylenediamines, para-aminophenols, ortho-aminophenols, heterocyclic bases, the addition salts thereof, and combinations thereof.

11. The hair color-altering composition of claim 9, wherein the fruit enzyme is present as part of a fruit extract selected from the group consisting of papaya extract, pineapple extract, and combinations thereof.

12. (canceled)

13. The hair color-altering composition of claim 9, wherein the composition has a pH of greater than about 7.

14. The hair color-altering composition of claim 9, further comprising an alkalizing agent selected from the group consisting of NH₄OH, monoethanolamine, and combinations thereof, wherein the alkalizing agent is present in an amount of greater than about 0 to about 20% by weight of the composition.

15. A kit comprising:
a. a first container comprising the hair color-altering composition of claim 9; and
b. a second container comprising a developer comprising hydrogen peroxide.

16-22. (canceled)

23. A method of altering the color of human hair, the method comprising applying to the hair color-altering composition of claim 1.

24. A method of altering the color of human hair, the method comprising applying to the hair the hair color-altering composition of claim 9.

25. (canceled)

26. The hair color-altering composition of claim 1, wherein the fruit enzyme is bromelain.

27. The hair color-altering composition of claim 1, wherein the fruit enzyme is papain.

28. The hair color-altering composition of claim 9, wherein the fruit enzyme is bromelain.

29. The hair color-altering composition of claim 9, wherein the fruit enzyme is papain.
